(19) United States
(12) Patent Application Publication
(10) Pub. No.: US 2024/0341804 A1
(43) Pub. Date: Oct. 17, 2024

(54) MULTIPLE CANNULA TROCAR ASSEMBLY

(71) Applicant: Alcon Inc., Fribourg (CH)

(72) Inventor: Paul R. Hallen, Colleyville, TX (US)

(21) Appl. No.: 18/606,003

(22) Filed: Mar. 15, 2024

Related U.S. Application Data
(60) Provisional application No. 63/495,578, filed on Apr. 12, 2023.

(51) Int. Cl. A61B 17/34; A61B 17/02

(52) U.S. Cl. CPC A61B 17/3421; A61B 17/0218; A61B 2017/3447

(57) ABSTRACT

A trocar assembly for eye surgery with a blade configured to accommodate multiple cannulas simultaneously. The blade may include one or more detents for retaining any cannulas in advance of cannula placement. Accommodating multiple cannulas in this manner facilitates new techniques for eye cannula placement in which trocar assembly disposal is minimized and efficiencies associated with tool change out for the surgery are enhanced.

### BACKGROUND

Vitrectomy is the removal of some or all of the vitreous humor from a patient's eye. In some cases, where the surgery is limited to removal of clouded vitreous humor, the vitrectomy may constitute the majority of the procedure. However, a vitrectomy may accompany surgery to repair a retina, to address a macular pucker or a host of other issues.

Regardless of the particulars, a few different incisions may be required for eye surgeries. For example, one incision may be made to provide access for a light instrument to illuminate the surgery. Another incision may be made for the vitrectomy probe or other surgical instrument. In some cases, another corresponding incision may be necessary to supply infusion fluid and maintain a proper balance within the eye in light of the withdrawn vitreous humor via the vitrectomy probe.

For each of these incisions, a cannula and trocar assembly may be used to make the incision and subsequently support the corresponding tool. Specifically, a blade of the trocar may be used to puncture the eye at the appropriate location, forming the incision. The blade may then be inserted into the eye until an under surface of a cannula surrounding the blade makes contact with the outer surface of the eye, generally at a scleral location.

With the cannula set, the trocar may be disposed of and the process repeated, (e.g., two more times with two more trocar and cannula assemblies). Regardless, the number of cannulas to be set as described, each cannula requires its own dedicated trocar for placement. The trocar blade may be under 1 mm (millimeter) in diameter and the cannula may be under 5 mm at its hub.

Unfortunately, the routine disposal of several trocars for almost all eye surgeries adds up to a significant amount of waste and wasted effort. Further, the disposal of trocars in this manner does not just increase the medical waste involved, but it also means the need for three times the amount of manufacturing effort devoted to high precision instruments.

### SUMMARY

A trocar assembly that accommodates multiple cannulas for eye surgery. The assembly includes a handle for surgeon manipulation and a trocar blade extending from the handle. The blade includes at least one detent extending from an outer surface thereof. A plurality of the cannulas are positioned about the trocar blade where at least one cannula of the plurality is retained by the at least one detent.

### BRIEF DESCRIPTION OF THE DRAWINGS

FIG. 1 is a perspective view of an embodiment of a multiple cannula trocar assembly for use in eye surgery.

FIG. 2 is an enlarged view of a needle of the multiple cannula trocar assembly.

FIG. 3A is an enlarged view of the needle of FIG. 2 during placement of a first cannula from the assembly.

FIG. 3B is an enlarged view of the needle of FIG. 2 during placement of a second cannula from the assembly.

FIG. 3C is an enlarged view of the needle of FIG. 2 during placement of a third cannula from the assembly.

FIG. 4 is a sectional overview of an eye during a vitrectomy surgical procedure facilitated by placement of multiple cannulas by the single trocar assembly.

FIG. 5 is a flow-chart summarizing an embodiment of employing a multiple cannula trocar assembly to facilitate eye surgery.

### DETAILED DESCRIPTION

In the following description, numerous details are set forth to provide an understanding of the present disclosure. However, it will be understood by those skilled in the art that the embodiments described may be practiced without these particular details. Further, numerous variations or modifications may be employed which remain contemplated by the embodiments as specifically described.

Embodiments are described with reference to certain types of surgical procedures. For example, a procedure in which vitreous humor is removed to address vitreous hemorrhage is illustrated. For such a procedure, embodiments of a multiple cannula trocar assembly may be utilized at various eye locations for cannula placement in support of a variety of different procedures. For example, a cannula placed by the assembly may facilitate positioning for a needle of a vitreous probe, a light source or an infusing instrument. Such procedures may be utilized to address retinal detachments, macular pucker, macular holes, vitreous floaters, diabetic retinopathy or a variety of other eye conditions. Regardless, so long as multiple cannulas may be positioned as indicated by way of the same trocar assembly, appreciable benefit may be realized. Further, while the trocar cannula is described in the context of use in the eye, it is to be understood that the trocar cannula may be used for any suitable body part where a cannula may be needed for a surgical procedure.

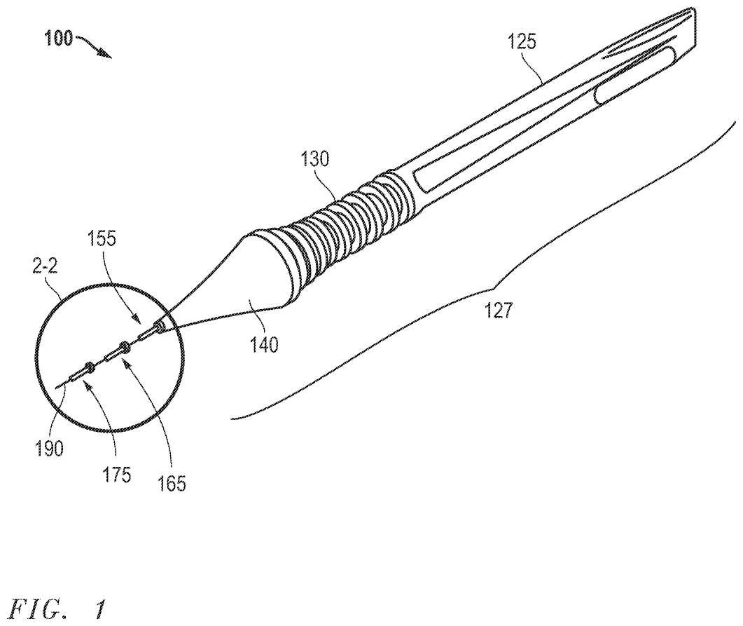

Referring now to FIG. 1, a perspective view of an embodiment of a multiple cannula trocar assembly 100 is shown for use in eye surgery. The assembly 100 includes a trocar blade 190 extending from a face 140 of the trocar body 127. The body 127 includes a conventional handle 125 and gripping region 130.

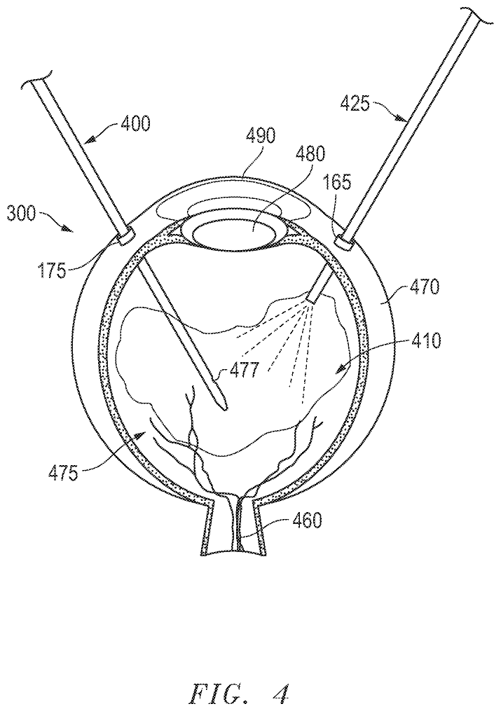

The trocar blade 190 is configured for making eye incisions. More specifically, an eye incision may be made with the blade 190 which may be left in place for delivery of a cannula (e.g., 175). Thus, eye surgery may be facilitated such as that depicted in FIG. 4. Regardless, for the embodiment illustrated, the blade 190 is configured to accommodate multiple cannulas 155, 165, 175. Thus, unlike a conventional trocar assembly that is thrown out after a single cannula placement, the single depicted assembly 100 may be used for multiple cannula placements to facilitate eye surgery. As a result, equipment costs and the surgical efficiencies of fewer tool change outs, may both be beneficially impacted.

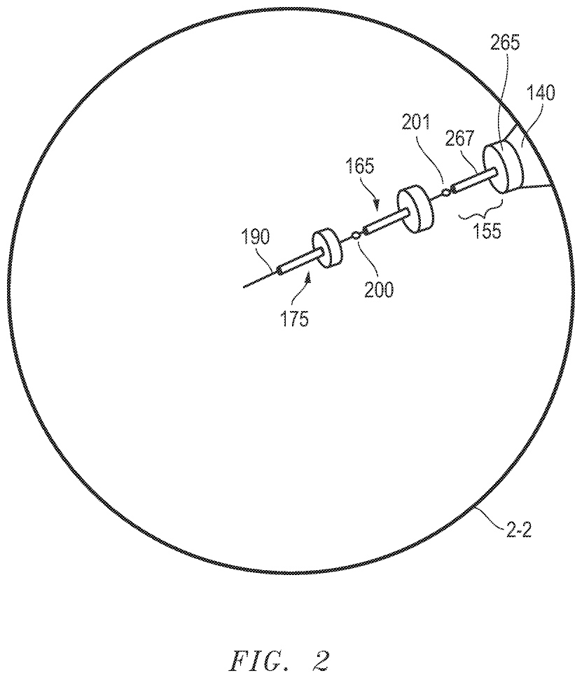

Referring now to FIG. 2 an enlarged view of a needle 190 of the multiple cannula trocar assembly 100 is shown taken from 2-2 of FIG. 1. This highlights three separate cannulas 155, 165, 175 being accommodated by the single needle 190. For sake of illustration, the cannulas 155, 165, 175 are shown spaced apart for visibility. However, in some embodiments, spacing between the cannulas 155, 165, 175 may be small (e.g., due to the presence of detents 200, 201 as described below).

As illustrated, each cannula 155, 165, 175 includes a conventional valve hub 265 and tubular cannula extension 267. Thus, with an inner diameter of the extensions 267 being slightly larger than the outer diameter of the blade 190, the blade 190 may accommodate multiple cannulas 155, 165, 175 in series as illustrated.

Of course, with multiple cannulas 155, 165, 175 on the blade 190, retention of cannulas not yet ready for placement at the eye may be of benefit (e.g., 155, 165). Thus, detents 200, 201 are provided. These detents 200, 201 may constitute any suitable form of mechanically raised profile at the outer surface of the needle 190. For sake of illustration, the size of the detents 200, 201 is slightly exaggerated. For example, the detents 200, 201 may be visibly imperceptible by the surgeon without close examination.

By way of example only, the assembly 100 of FIG. 1 may include a 25 gauge cannula 155, 165, 175 that is paired with a 26 gauge trocar blade or needle 190. In such a circumstance, the inner diameter of the cannula 155, 165, 175 is generally sufficient to accommodate the needle 190 through the extension 167 and hub 265. However, a raised profile in the form of detents 200, 201 extending the blade gauge to about 24 is also substantial enough to prevent accidental movement of a cannula 155, 165 toward the distal end of the needle 190 (e.g., opposite the handle 125) (e.g., through frictional engagement between an outer surface of the detent and an inner surface of the cannula).

Notice that for the embodiment shown, no detents 200, 201 are depicted distal of the first cannula 175 to be placed. Rather, for this cannula 175, a conventional outer surface of the needle 190 may be utilized without concern over more proximal cannulas 155, 165 coming into contact with or unintentionally advancing this first cannula 175 in a premature manner. Of course, in another embodiment, additional detents 200, 201 may be positioned distal to this first cannula 175 for added stability. On the other hand, in yet another embodiment, detents 200, 201 may be limited to a location between this first cannula 175 for placement and the next (e.g., 165). That is, depending on the profile and nature of the detents 200, 201, detents just proximal of the first cannula 175 may serve as a sufficient safeguard to the potential for the other cannulas 165, 155 to advance.

The detents 200, 201 may be of any suitably minimal profile that is sufficient to discourage cannulas 155, 165, 175 from premature advancement (e.g., through frictional engagement between the detents and the inner surface of the cannulas). At the same time, the detents 200, 201 are small enough to allow a minimal amount of force to advance a cannula 155, 165, 175 in a distal direction away from the handle 125 of FIG. 1. Indeed, this may be less than the minimal force employed by the surgeon in setting a cannula 155, 165, 175. For example, a surgeon performing an eye surgery as illustrated in FIG. 4, may achieve this advancement through manual manipulation alone.

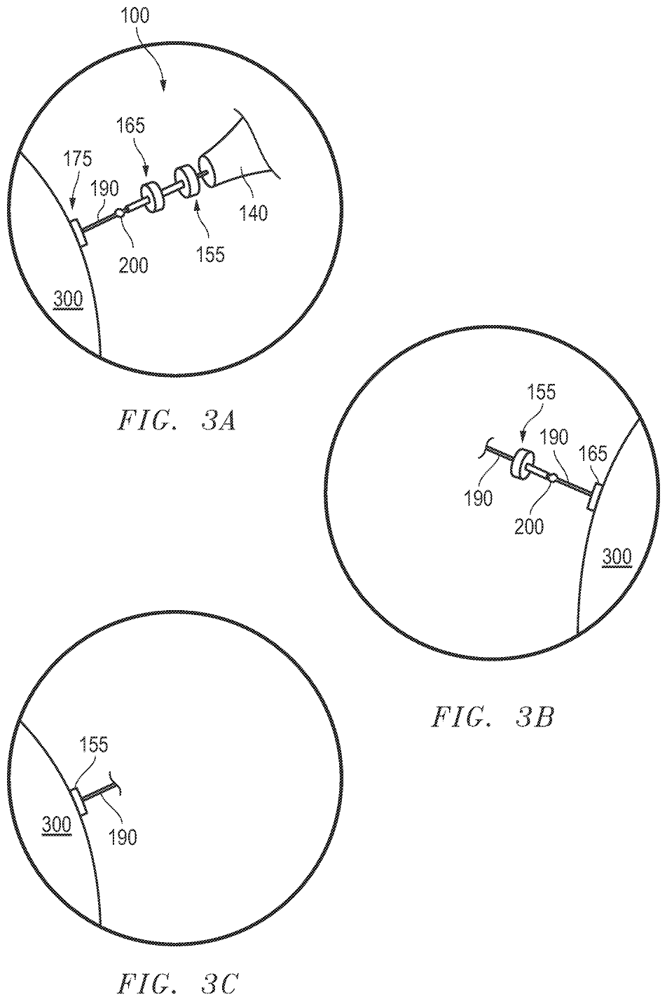

Referring now to FIGS. 3A-3C, an enlarged view of the needle 190 is illustrated during placement of the cannulas 155, 165, 175 of FIG. 2. In this view, the placement of the first cannula 175 is illustrated at FIG. 3A. Notice in this view that detents 200 are illustrated only above or proximal to the set cannula 175 at the eye 300 of a patient. For this embodiment, the placed cannula 175 is conventional in its relationship with the needle 190 such that it is allowed a degree of flexibility in movement along the needle 190. The detents 200 depicted are limited to a location that is proximal or above this first cannula 175 which is sufficient to avoid unintentional contact or advancing as a result of any physical interference from the more proximal cannulas 165, 155.

The illustrated detents 200 are also depicted as more of a smooth raised bump, ring or other slight protrusion to present a raised profile at the outer surface of the needle 190 for temporarily keeping more proximal cannulas 165, 155 in position above the first cannula 175. However, in other embodiments, the detents 200 may be deflectable, retractable or reciprocating arms. Whatever the case, unintentional advancement past the detents 200 during setting of the first cannula 175 is prevented.

Referring now to FIG. 3B, an enlarged view of the needle 190 of FIG. 2 is shown during placement of a second cannula 165 from the assembly 100 of FIG. 1. In this view, this second cannula 165 has been advanced past the detents 200 and positioned in place at the eye 300 to facilitate eye surgery as illustrated in FIG. 4. As noted above, this may be achieved through minimal force applied manually by the surgeon or with other aids such as a cannula setting implement.

Referring now to FIG. 3C, an enlarged view of the needle 190 of FIG. 2 is shown during placement of a third cannula 155 from the assembly 100 of FIG. 1. In this view it is apparent that three different cannulas 155, 165, 175 have been placed in succession from the same assembly needle 190. This means that three different surgical access points have been provided as shown in FIG. 4 without the need for any tool changeout or waste. Rather, the same needle 190 being utilized on the same patient and eye 300 is configured to avoid the need for the introduction, discarding and re-introduction of needle after needle. Instead, a single assembly 100 as shown in FIG. 1 may be utilized for placement of all three cannulas 155, 165, 175. An exemplary three cannula assembly is discussed here given that this is often the common practice for eye surgery. Of course, a two cannula, four cannula or other number of multiple cannula embodiment may be employed where practical given the surgical particulars.

Referring now to FIG. 4, a sectional overview of the eye 300 during a vitrectomy surgical procedure is illustrated. This procedure is facilitated by prior placement of multiple cannulas (165, 175) by the single trocar assembly 100 of FIG. 1. In this embodiment, the last cannula 155 of FIGS. 1, 2 and 3A, is positioned at a portion of the eye 300 at an opposite side that is not visible in the illustrated depiction.

The cannulas 165, 175 shown in FIG. 4 are placed to facilitate guided support to a vitrectomy probe 400 and a light source 425. The cannulas 165, 175 are relatively short in length to help avoid risk of damage to the optic nerve 460, retina 475 and other more delicate features at the back of the eye 300 from a cannula extension 267 (see FIG. 2). Similarly, the trocar blade or needle 190, and the instruments 400, 425 here are configured to interface with the cannulas 165, 175 that also prohibits reaching too deep into the eye 300.

With the support and guidance of the placed cannulas 165, 175, the surgery may securely proceed. In the illustrated example, the needle of a vitrectomy probe 400 is inserted through one of the cannulas 175 and directed toward a region 410 where vitreous humor is to be removed. Specifically, a suction is applied and the port 477 is used for the uptake of the vitreous humor or other substances. For example, in the procedure illustrated, a hemorrhage may be taking place in the region 410 such that blood is drawn into the port 477 along with the vitreous humor.

The surgery illustrated also includes a light instrument 425 reaching into the eye 300 through another cannula 165. In both cases, the cannulas 165, 175 are positioned in an offset manner at the sclera 470. In this way, the more delicate cornea 490 and lens 480 may be avoided.

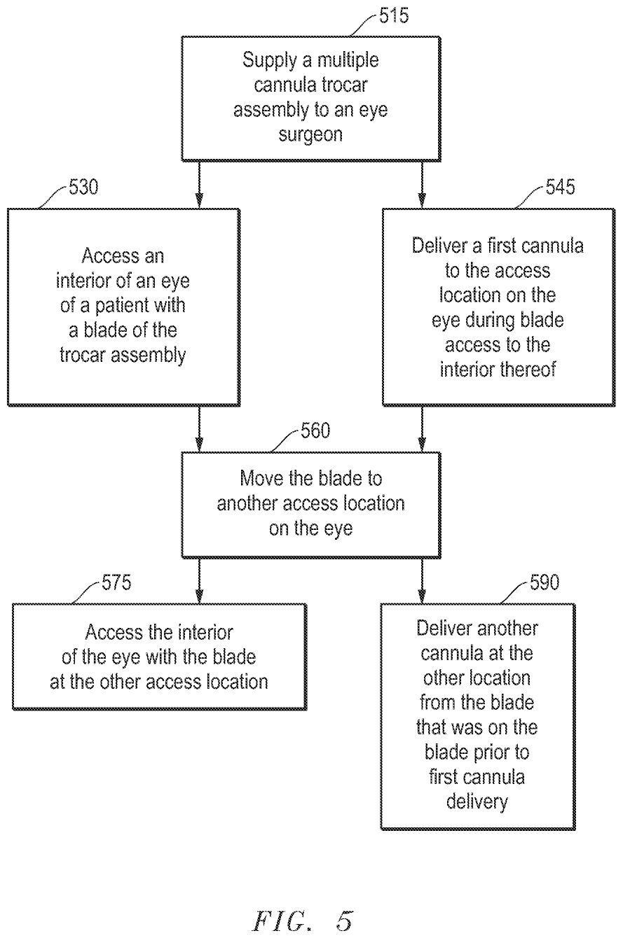

Referring now to FIG. 5, a flow-chart summarizing an embodiment of employing a multiple cannula trocar assembly to facilitate eye surgery is illustrated. With a multiple cannula, single trocar assembly in the hands of an eye surgeon as indicated at 515, a blade of the assembly may be used to access an interior of an eye and deliver a first cannula to the access location on the eye (see 530, 545). Notably however, the blade may be re-used without time spent reloading with another cannula. Instead, the blade is already outfitted with another cannula. Therefore, as noted at 560, the blade may be moved to another location of the eye to provide access to the interior from another access location (see 575). With the blade in place, another cannula may be delivered to this other access location (see 590).

For example, a first detent may be sized to frictionally engage a first cannula of a plurality of cannulas relative to the trocar blade as the trocar blade is inserted into a body to place the first cannula in the body. The first detent may have an outer diameter sized to frictionally engage with an inner surface of the first cannula. In some embodiments, the frictional engagement holds the first cannula in place relative to the trocar blade during insertion of the first cannula into the body. The trocar blade may include a second detent located on the trocar blade at a location closer to the handle than the first detent, and the second detent may frictionally engage a second cannula. Additional detents may similarly retain additional cannulas along the trocar blade. In some embodiments, the second detent is sized to allow the second cannula to be advanced over the second detent to stage at the first detent through manual manipulation by a user. In some embodiments, the first detent is sized to frictionally engage the second cannula relative to the trocar cannula as the trocar cannula is re-inserted into the body to place the second cannula in the body. The second cannula may then remain in the body as the trocar blade is withdrawn (e.g., interference between the outer surface of the cannula and the body portion in contact with the cannula may be greater than the frictional interference between the first detent and the cannula inner surface). In some embodiments, the user may also assist the cannula remaining in the body as the trocar blade is withdrawn by applying pressure to a hub (e.g., through the user's finger) as the trocar blade is withdrawn.

Embodiments described hereinabove include tools and techniques that allow for the delivery of multiple eye cannulas from a single trocar assembly. In this manner, trocar assemblies may avoid being disposed of between multiple uses on the same eye. Indeed, the blade of the assembly may be preloaded such that stopping the procedure to reload the blade with another cannula is avoidable. Instead, after placement of an initial cannula with the assembly, the surgeon may move directly on to the next cannula placement. Thus, both waste and surgical efficiencies may be enhanced.

The preceding description has been presented with reference to several embodiments. However, other embodiments and/or features of the embodiments disclosed but not detailed hereinabove may be employed. Furthermore, persons skilled in the art and technology to which these embodiments pertain will appreciate that still other alterations and changes in the described structures and methods of operation may be practiced without meaningfully departing from the principle and scope of these embodiments. Additionally, the foregoing description should not be read as pertaining only to the precise structures described and shown in the accompanying drawings, but rather should be read as consistent with and as support for the following claims, which are to have their fullest and fairest scope.

1. A trocar assembly for placement of multiple cannulas, the assembly comprising:
a body of the assembly with a handle;
a trocar blade extending from the body, the blade including at least one detent extending from an outer surface thereof; and
a plurality of the cannulas about the trocar blade, wherein the at least one detent is sized to frictionally engage at least one cannula of the plurality of cannulas relative to the trocar blade as the trocar blade is inserted into a body to place the at least cannula in the body.

2. The trocar assembly of claim 1, wherein the at least one detent comprises a first detent that has an outer diameter sized to frictionally engage with an inner surface of the at least one cannula.

3. The trocar assembly of claim 2, wherein the frictional engagement holds the at least one cannula in place relative to the trocar blade during insertion of the at least one cannula into the body.

4. The trocar assembly of claim 2, wherein the at least one detent comprises a second detent located on the trocar blade at a location closer to the handle than the first detent, and wherein the second detent is configured to frictionally engage a second cannula of the at least one cannula.

5. The trocar assembly of claim 4, wherein the second detent is sized to frictionally engage but yet allow the second cannula to be advanced over the second detent to stage at the first detent through manual manipulation by a user.

6. The trocar assembly of claim 5, wherein the first detent is sized to frictionally engage the second cannula relative to the trocar cannula as the trocar cannula is re-inserted into the body to place the second cannula in the body.

7. The assembly of claim 1 wherein each cannula of the plurality comprises:
a hub for securing at an outer surface location of the eye; and
a tubular extension coupled to the hub and for providing access to an interior of the body.

8. A method of positioning multiple cannulas to support a surgical procedure, the method comprising:
penetrating a surface of the body at a first location with a trocar blade accommodating multiple cannulas;
delivering a first cannula of the multiple cannulas from the blade to the first location;
moving the trocar blade to another location;
penetrating a surface of the body at the other location with the trocar blade; and
delivering a second cannula of the multiple cannulas from the blade to the second location.

9. The method of claim 8 further comprising accessing an interior of the body with an extension of one of the cannulas.

10. The method of claim 8 where in the blade includes at least one detent for retaining the second cannula in advance of the moving of the blade to another location.

11. The method of claim 8 further comprising performing a surgical procedure through the cannulas, the procedure comprising:
positioning a first surgical tool through the first cannula and to the interior; and
positioning a second surgical tool through the second cannula and to the interior.

12. The method of claim 8, wherein delivering the first cannula comprises frictionally engaging the first cannula through a first detent on the trocar blade as the trocar blade is inserted into a body to place the first cannula in the body.

13. The method of claim 12, wherein before delivering the second cannula, the method further comprises:
frictionally engaging the second cannula through a second detent on the trocar blade at a location closer to the handle than the first detent.

14. The method of claim 13, further comprising advancing the second cannula over the second detent to stage at the first detent through manual manipulation by a user.

15. The method of claim 14, wherein delivering the second cannula comprises frictionally engaging the second cannula relative to the trocar cannula as the trocar cannula is re-inserted into the body to deliver the second cannula to the second location.
